(19) United States
(12) Patent Application Publication
(10) Pub. No.: US 2003/0134002 A1
(43) Pub. Date: Jul. 17, 2003

(54) Method for treating visual impairment through the prophylactic administration of a Morinda citrifolia-based naturaceutical

(76) Inventors: Claude Jarakae Jensen, Cedar Hills, UT (US); Enrique Ocampo, El Paso, TX (US)

Correspondence Address: KIRTON & McCONKIE, 1800 Eagle Gate Tower, 60 East South Temple, Salt Lake City, UT, 84111, US

(21) Appl. No.: 10/285,711

(22) Filed: Nov. 1, 2002

Related U.S. Application Data
(60) Provisional application No. 60/335,322, filed on Nov. 2, 2001.

(51) Int. Cl. A61K 35/78

(52) U.S. Cl. 424/765; 424/769

(57) ABSTRACT

Implementation of the present invention takes place in association with the utilization of one or more processed products produced from the Indian Mulberry plant, scientifically known as Morinda citrifolia L., to treat one or more eye disorders that affect vision, such as glaucoma, diabetic retinopathy, retinitis pigmentosa, cataracts, age-related macular degeneration, night blindness, color blindness, and other related conditions. The processed Morinda citrifolia products from the Indian Mulberry plant may be in the form of a dietary supplement, eye drops, or in another suitable form.

### RELATED APPLICATIONS

This application claims priority to United States Provisional Application Serial No. 60/335,322 filed Nov. 2, 2001, and entitled, “Methods for Treating Visual Impairments.”

### BACKGROUND

1. Field of the Invention

The present invention relates to methods and naturaceutical formulations and substances for treating and preventing ocular or visual impairments. Specifically, the present invention relates to Morinda citrifolia-based methods and naturaceutical formulations and substances for treating pre-existing ocular impairments, as well as to Morinda citrifolia-based methods and naturaceutical formulations and substances for preventing the onset or reducing the onset potential of future or additional ocular impairments. The present invention is particularly suited for treatment and prevention of ocular impairments as commonly experienced in mammals, and particularly humans.

2. Background of the Invention and Related Art

The eye is an organ that collects light and turns it into electronic messages that are sent to the brain. The brain then turns those signals into a picture for an individual to see. Since individuals have two eyes, two pictures are usually created, which accounts for depth of vision. Most of depth of vision occurs from judging the relative size of the objects seen.

The eye includes several intricate parts or components. The eyelids hold the lashes, keep the eye moist, and shield it from intense light. The conjunctiva is a membrane that covers most of the eyeball and allows the lids to gently glide over the eye. The clear cornea covers the iris, and works like a watch-face for the eye. It allows a small amount of light to enter the eye through the pupil. Then, along with the natural lens, it acts like a camera-lens and focuses the image onto the retina. The retina is like the film in a “ocular” camera. It lines the inside of the eye, and is mostly clear. The retina has very few blood vessels that would disturb the retinal picture. Since the retina has so few blood vessels and does a lot of work, it needs to be nourished by a blood vessel layer beneath it. This sub-layer blood vessel is called the choroid or uvea.

Not only does the choroid feed the retina, but it also contains pigment cells called melanocytes. These cells and their output product “melanin” absorb extra light that may distort the retinal picture. Melanin is also thought to protect against the development of age-related macular degeneration.

Various eye disorders exist that negatively affect vision. For example, with dry macular degeneration the retinal and choroidal blood vessels slowly disappear, as does the central retina. Alternatively, “wet” macular degeneration, abnormal blood vessels grow under the macular retina, leak and cause scarring in the central retina.

Visual impairments are caused by a variety of factors, including aging, disease, inherited attributes and accidents. For example, blindness and/or visual impairment may be attributed to glaucoma, diabetic retinopathy, retinitis pigmentosa, cataracts, age-related macular degeneration, and other such eye conditions or disorders.

Glaucoma is an eye condition that typically causes blindness without any symptoms or pain. The optic nerve of the eye is damaged due to pressure from a build up of fluid. The pressure typically causes clouding and may result in blindness. An eye examination is typically required to determine whether or not glaucoma is present.

Diabetic retinopathy is a disease of the eye that is caused when the small blood vessels in the retina begin to weaken or become blocked. The disease's effect on vision can range from a distortion similar to looking through water, to dark spots, to clouding, or to cobweb-like strands that distort vision. The retina typically becomes detached resulting in severe vision loss or blindness. For people with diabetes, controlling blood sugar levels, by diet, and by taking insulin, is typically used to treat the risk of this disease.

Retinitis Pigmentosa is the name given to a group of eye diseases that are genetic. Retinitis Pigmentosa affects the part of the eye that is sensitive to light and typically develops slowly so that severe vision loss may occur over the span of many years. Symptoms typically include night blindness and a loss of side vision, which worsens over time until all side vision is lost. Retinitis Pigmentosa may lead to total blindness.

A cataract is a clouding of the lens in the eye, usually associated with aging. Typically, surgery is the method employed to remove the damaged lens in order to restore sight to the person with cataracts. While this method may be effective, the surgery procedure presents risks to the individual patients.

Macular degeneration affects central vision of the individual and the majority of individuals with this condition are typically either partially sighted or legally blind. Activities that require good central vision such as reading, writing and carrying out certain domestic tasks are all affected. Many macular degeneration patients require assistance in performing daily activities. Sometimes, their visual handicap necessitates admission to a nursing home. The social cost of this handicap is enormous in both personal and social terms. When both eyes are affected, the individuals experience a serious loss in their quality of life and independence.

One such form of macular degeneration is the neovascular “wet” form, which is responsible for a large majority of severe loss of vision for individuals. This form is usually associated with aging, but other diseases may cause wet macular degeneration including high myopia and some intraocular infections like histoplasmosis. The only proven treatment for wet macular degeneration is laser photocoagulation, which is an option for only 10-15% of the individuals. This is typically because most patients have the occult or mixed form of the disorder where the doctor cannot see the entire neovascular lesion on the fluorescein angiogram. Laser success is dependent upon the doctor being able to see the entire area of new blood vessel formation, “neovascularization,” and those that are treated typically experience recurrences within five years of the laser treatment. Furthermore, another problem with the laser treatment is that it does not fix the macular problem that caused the new blood vessels to grow in the first place.

While improvements in alternatives for treating patients with eye disorders have occurred in recent decades, researchers are continually attempting to obtain improved methods of treatment, as some of the treatments are undesirable. Furthermore, some eye disorders currently have no cure or treatment.

Accordingly, it would be an improvement in the art to augment or even replace the treatments currently used to provide increased results in treating visual impairments of individuals.

### SUMMARY AND OBJECTS OF THE INVENTION

The present invention relates to methods for treating visual impairment. In particular, the present invention relates to providing a treatment for visual impairments that includes one or more processed products as derived from the Indian Mulberry plant, scientifically known as Morinda citrifolia L.

Implementation of the present invention takes place in association with the utilization of a naturaceutical formulation comprising a Morinda citrifolia product, and particularly a fruit or puree juice or a leaf extract, from the Indian Mulberry plant, scientifically known as Morinda citrifolia L., to treat one or more eye disorders that affect vision, such as glaucoma, diabetic retinopathy, retinitis pigmentosa, cataracts, age-related macular degeneration, night blindness, color blindness, and others. The processed Morinda citrifolia product may be in the form of a dietary supplement, eye drops, or in any other suitable form.

Therefore, it is an object of some embodiments of the present invention to provide a naturaceutical formulation for treating and preventing visual impairments.

It is another object of some embodiments of the present invention to provide a naturaceutical formulation comprising one or more processed Morinda citrifolia products as an active ingredient in treating and preventing visual impairments.

It is still another object of some embodiments of the present invention to provide one or more methods of administering the present invention naturaceutical formulation to be internalized by an individual to effectuate treatment of a visual impairment.

It is a further object of some embodiments of the present invention to treat pre-existing visual impairments, as well as to prevent the onset of visual impairments.

In accordance with the invention as embodied and broadly described herein, the present invention features a naturaceutical formulation comprising one or more processed Morinda citrifolia products and other ingredients as or if desired. The present invention also features several methods of administering the naturaceutical formulation, such as orally through oral ingestion or by means of an eye drop composition or solution.

In accordance with the present invention, the naturaceutical formulation may exist or be manufactured in any suitable form for internalization into the body of the individual undergoing treatment. For example, in one exemplary embodiment, the naturaceutical may exist in the form of eye drops or an eye drop solution. In another exemplary embodiment, the naturaceutical may exist as a dietary supplement. Still other forms or embodiments capable of introducing the naturaceutical formulation into the body for internalization by the individual for the purpose of treating one or more eye disorders are contemplated herein. The amount or dose of the naturaceutical formulation used per treatment may depend on various factors, including the type of eye disorder, the physical characteristics of the patient, etc.

The use of processed Morinda citrifolia has proven to be advantageous in treating a variety of eye disorders by providing improved results. The processed Morinda citrifolia includes antibacterial properties, particularly useful in fighting, for example, Pseudomonas and Conjunctivitis infections. Furthermore, the use of processed Morinda citrifolia has proven to inhibit, prevent, and reverse macular degeneration.

Thus, implementation of the present invention embraces several methods for improved treatment of visual impairment. In particular, the present invention relates to providing a treatment for one or more eye disorders that includes juice from the Indian Mulberry plant, scientifically known as Morinda citrifolia L.

### DETAILED DESCRIPTION OF THE PREFERRED EMBODIMENTS

It will be readily understood that the components of the present invention, as generally described herein, could be arranged and designed in a wide variety of different configurations. Thus, the following more detailed description of the embodiments of the system and method of the present invention is not intended to limit the scope of the invention, as claimed, but is merely representative of the presently preferred embodiments of the invention.

The presently preferred embodiments of the invention will be best understood by separating the description into sections, the first pertaining to a general discussion regarding Morinda citrifolia, including its origins, processing techniques, and health benefits, and the methods employed to produce and manufacture the processed Morinda citrifolia products used as key ingredients in the naturaceutical formulations described herein; and the second being a more detailed and specific discussion on the Morinda citrifolia-based methods and naturaceutical formulations or compositions used to treat and prevent visual impairments and their associated symptoms or conditions, wherein such treatment methods involve the prophylactic administration of the processed Morinda citrifolia products as embodied and described herein.

### General Discussion of Morinda Citrifolia and the Methods Used to Produce Processed Morinda Citrifolia Products

The Indian Mulberry or Noni plant, known scientifically as Morinda Citrifolia L. (Morinda citrifolia), is a shrub or small tree up to 10 m in height. The leaves are oppositely arranged with an elliptic to ovate form. The small white flowers are contained in a fleshy, globose, head-like cluster. The fruits are large, fleshy, and ovoid. At maturity, they are creamy-white and edible, but have an unpleasant taste and odor. The plant is native to Southeast Asia and has spread in early times to a vast area from India to eastern Polynesia. It grows randomly in the wild, and it has been cultivated in plantations and small individual growing plots. The Morinda citrifolia flowers are small, white, three to five lobed, tubular, fragrant, and about 1.25 cm long. The flowers develop into compound fruits composed of many small drupes fused into an ovoid, ellipsoid or roundish, lumpy body, with waxy, white, or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged 2-celled stones, each containing four seeds.

When fully ripe, the fruit has a pronounced odor like rancid cheese. Although the fruit has been eaten by several nationalities as food, the most common use of the Morinda citrifolia plant was as a red and yellow dye source. Recently, there has been an interest in the nutritional and health benefits of the Morinda citrifolia plant, further discussed below.

Because the Morinda citrifolia fruit is for all practical purposes inedible, the fruit must be processed in order to make it palatable for human consumption and included in food products used to treat visual impairments and its related symptoms. Processed Morinda citrifolia fruit juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in another food product, frozen or pasteurized. In some embodiments, the juice and pulp can be pureed into a homogenous blend to be mixed with other ingredients. Other process include freeze drying the fruit and juice. The fruit and juice can be reconstituted during production of the final juice product. Still other processes include air drying the fruit and juices, prior to being masticated.

The present invention utilizes the fruit juice, the puree, and the oil extracted from the Morinda Citrifolia plant. In a currently preferred process of producing Morinda citrifolia fruit juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from 0 to 14 days, with most fruit being held from 2 to 3 days. The fruit is ripened or aged by being placed on equipment so it does not contact the ground. It is preferably covered with a cloth or netting material during aging, but can be aged without being covered. When ready for further processing the fruit is light in color, from a light green, light yellow, white or translucent color. The fruit is inspected for spoilage or for excessively green color and hard firmness. Spoiled and hard green fruit is separated from the acceptable fruit.

The ripened and aged fruit is preferably placed in plastic lined containers for further processing and transport. The containers of aged fruit can be held from 0 to 30 days. Most fruit containers are held for 7 to 14 days before processing. The containers can optionally be stored under refrigerated conditions prior to further processing. The fruit is unpacked from the storage containers and is processed through a manual or mechanical separator. The seeds and peel are separated from the juice and pulp.

The juice and pulp can be packaged into containers for storage and transport. Alternatively, the juice and pulp can be immediately processed into a finished juice product in concentrate or dilute (such as with water or other fruit juices) form. The containers can be stored in refrigerated, frozen, or room temperature conditions.

The Morinda citrifolia juice and pulp are preferably blended in a homogenous blend, after which they may be mixed with other ingredients, such as flavorings, sweeteners, nutritional ingredients, botanicals, and colorings. The finished juice product is preferably heated and pasteurized at a minimum temperature of 181° F. (83° C.) or higher up to 212° F. (100° C.).

Another product manufactured is Morinda citrifolia puree and puree juice, in either concentrate or diluted form. Puree is essentially the pulp a separated from the seeds and is different than the fruit juice product described herein.

Each product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp may be further processed by separating the pulp from the juice through filtering equipment. The filtering equipment may include a centrifuge decanter, a screen filter with a size from 1 micron up to 2000 microns, more preferably less than 500 microns, a filter press, reverse osmosis filtration., and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered at least once and up to 10 times to remove any juice from the pulp. The wet pulp typically has a fiber content of 10 to 40 percent by weight. The wet pulp may be pasteurized at a temperature of 181° F. (83° C.) minimum and then packed in drums for further processing or made into a high fiber product.

Drying may further process the wet pulp. The methods of drying may include freeze-drying, drum drying, tray drying, sun drying, and spray drying. The dried Morinda citrifolia pulp may include a moisture content in the range from 0.1 to 15 percent by weight and more preferably from 5 to 10 percent by weight. The dried pulp preferably has a fiber content in the range from 0.1 to 30 percent by weight, and more preferably from 5 to 15 percent by weight.

The high fiber product may include wet or dry Morinda citrifolia pulp, supplemental fiber ingredients, water, sweeteners, flavoring agents, coloring agents, and/or nutritional ingredients. The supplemental fiber ingredients may include plant based fiber products, either commercially available or developed privately. Examples of some typical fiber products are guar gum, gum arabic, soybean fiber, oat fiber, pea fiber, fig fiber, citrus pulp sacs, hydroxymethylcellulose, cellulose, seaweed, food grade lumber or wood pulp, hemicellulose, etc. Other supplemental fiber ingredients may be derived from grains or grain products. The concentrations of these other fiber raw materials typically range from 0 up to 30 percent, by weight, and more preferably from 10 to 30 percent by weight.

Typical sweeteners may include, but are not limited to, natural sugars derived from corn, sugar beet, sugar cane, potato, tapioca, or other starch-containing sources that can be chemically or enzymatically converted to crystalline chunks, powders, and/or syrups. Also sweeteners can consist of artificial or high intensity sweeteners, some of which are aspartame, sucralose, stevia, saccharin, etc. The concentration of sweeteners may be between from 0 to 50 percent by weight, of the formula, and more preferably between about 1 and 5 percent by weight.

Typical flavors can include, but are not limited to, artificial and/or natural flavor or ingredients that contribute to palatability. The concentration of flavors may range, for example, from 0 up to 15 percent by weight, of the formula. Colors may include food grade artificial or natural coloring agents having a concentration ranging from 0 up to 10 percent by weight, of the formula.

Typical nutritional ingredients may include vitamins, minerals, trace elements, herbs, botanical extracts, bioactive chemicals and compounds at concentrations from 0 up to 10 percent by weight. Examples of vitamins one can add to the fiber composition include, but are not limited to, vitamins A, B1 through B12, C, D, E, Folic Acid, Pantothenic Acid, Biotin, etc. Examples of minerals and trace elements one can add to the fiber composition include, but are not limited to, calcium, chromium, copper, cobalt, boron, magnesium, iron, selenium, manganese, molybdenum, potassium, iodine, zinc, phosphorus, etc. Herbs and botanical extracts include, but are not limited to, alfalfa grass, bee pollen, chlorella powder, Dong Quai powder, Ecchinacea root, Gingko Biloba extract, Horsetail herb, Indian mulberry, Shitake mushroom, spirulina seaweed, grape seed extract, etc. Typical bioactive chemicals may include, but are not limited to, caffeine, ephedrine, L-carnitine, creatine, lycopene, etc.

The juice and pulp can be dried using a variety of methods. The juice and pulp mixture can be pasteurized or enzymatically treated prior to drying. The enzymatic process begins with heating the product to a temperature between 75° F. and 135° F. It is then treated with either a single enzyme or a combination of enzymes. These enzymes include, but are not limited to, amylase, lipase, protease, cellulase, bromelin, etc. The juice and pulp may also be dried with other ingredients, such as those described above in connection with the high fiber product. The typical nutritional profile of the dried juice and pulp is 1 to 20 percent moisture, 0.1 to 15 percent protein, 0.1 to 20 percent fiber, and the vitamin and mineral content.

The filtered juice and the water from washing the wet pulp are preferably mixed together. The filtered juice may be vacuum evaporated to a brix of 40 to 70 and a moisture of 0.1 to 80 percent, more preferably from 25 to 75 percent. The resulting concentrated Morinda citrifolia juice may or may not be pasteurized. For example, the juice would not be pasteurized in circumstances where the sugar content or water activity was sufficiently low enough to prevent microbial growth. It is packaged for storage, transport and/or further processing.

The processed Morinda citrifolia product may also exist as a dietary fiber produced from the fruit puree. Still further, the processed Morinda citrifolia product may also exist in oil form, such as an oil extract. The Morinda citrifolia oil typically includes a mixture of several different fatty acids as triglycerides, such as palmitic, stearic, oleic, and linoleic fatty acids, and other fatty acids present in lesser quantities. In addition, the oil preferably includes an antioxidant to inhibit spoilage of the oil. Conventional food grade antioxidants are preferably used.

In still another exemplary embodiment, a preferred embodiment, the processed Morinda citrifolia product may comprise an extract from the leaves of the Indian Mulberry plant in the form of a processed Morinda citrifolia leaf hot water extract, a processed Morinda citrifolia leaf ethanol extract, and a processed Morinda citrifolia leaf steam distillation extract.

First, frozen Morinda citrifolia leaves were defrosted and chopped into small pieces at the room temperature. Distilled water, five times the volume of the chopped leaves, was added and hot water extraction was conducted for an hour. Then the solids in the solution were removed by centrifugation and the supernatant obtained was freeze-dried as a processed Morinda citrifolia leaf hot water extract.

Second, frozen Morinda citrifolia leaves were defrosted and chopped into small pieces at the room temperature. Distilled water, five times the volume of the chopped leaves, was added, and agitated at 40° C. while extraction was conducted for one hour. After removing solid objects, ethanol was removed under decreasing pressure (rotary evaporator). Solids produced were removed with a fiberglass filter. The resulting supernatant was freeze dried as a processed Morinda citrifolia leaf ethanol extract.

Third, frozen Morinda citrifolia leaves were defrosted and chopped into small pieces at room temperatures. The chopped leaves were steam distilled using a subcritical steam distillation apparatus. Then the solids in the solution were removed by centrifugation and the supernatant obtained was freeze dried as a processed Morinda citrifolia leaf steam distilled extract. Other methods or procedures not specifically recited herein may be used to extract out the active ingredients found in the processed Morinda citrifolia leaf extracts, as will be apparent to one ordinarily skilled in the art. Therefore, the methods and procedures that are specifically recited herein are not intended to be limiting in any way.

The Morinda citrifolia plant is rich in natural ingredients. Those ingredients that have been discovered include: (from the leaves): alanine, anthraquinones, arginine, ascorbic acid, aspartic acid, calcium, beta-carotene, cysteine, cystine, glycine, glutamic acid, glycosides, histidine, iron, leucine, isoleucine, methionine, niacin, phenylalanine, phosphorus, proline, resins, riboflavin, serine, beta-sitosterol, thiamine, threonine, tryptophan, tyrosine, ursolic acid, and valine; (from the flowers): acacetin-7-o-beta-d(+)-glucopyranoside, 5,7-dimethyl-apigenin-4′-o-beta-d(+)-galactopyranoside, and 6,8-dimethoxy-3-methylanthraquinone-1-o-beta-rhamnosyl-glucopyranoside; (from the fruit): acetic acid, asperuloside, butanoic acid, benzoic acid, benzyl alcohol, 1-butanol, caprylic acid, decanoic acid, (E)-6-dodeceno-gamma-lactone, (Z,Z,Z)-8,11,14-eicosatrienoic acid, elaidic acid, ethyl decanoate, ethyl hexanoate, ethyl octanoate, ethyl palmitate, (Z)-6-(ethylthiomethyl)benzene, eugenol, glucose, heptanoic acid, 2-heptanone, hexanal, hexanamide, hexanedioic acid, hexanoic acid (hexoic acid), 1-hexanol, 3-hydroxy-2-butanone, lauric acid, limonene, linoleic acid, 2-methylbutanoic acid, 3-methyl-2-buten-1-ol, 3-methyl-3-buten-1-ol, methyl decanoate, methyl elaidate, methyl hexanoate, methyl 3-methylthio-propanoate, methyl octanoate, methyl oleate, methyl palmitate, 2-methylpropanoic acid, 3-methylthiopropanoic acid, myristic acid, nonanoic acid, octanoic acid (octoic acid), oleic acid, palmitic acid, potassium, scopoletin, undecanoic acid, (Z,Z)-2,5-undecadien-1-ol, and vomifol; (from the roots): anthraquinones, asperuloside (rubichloric acid), damnacanthal, glycosides, morindadiol, morindine, morindone, mucilaginous matter, nor-damnacanthal, rubiadin, rubiadin monomethyl ether, resins, soranjidiol, sterols, and trihydroxymethyl anthraquinone-monomethyl ether; (from the root bark): alizarin, chlororubin, glycosides (pentose, hexose), morindadiol, morindanigrine, morindine, morindone, resinous matter, rubiadin monomethyl ether, and soranjidiol; (from the wood): anthragallol-2,3-dimethylether; (from the tissue culture): damnacanthal, lucidin, lucidin-3-primeveroside, and morindone-6beta-primeveroside; (from the plant): alizarin, alizarin-alpha-methyl ether, anthraquinones, asperuloside, hexanoic acid, morindadiol, morindone, morindogenin, octanoic acid, and ursolic acid.

Recently, as mentioned, many health benefits have been discovered stemming from the use of products containing Morinda citrifolia. One benefit of Morinda citrifolia is found in its ability to isolate and produce Xeronine, which is a relatively small alkaloid physiologically active within the body. Xeronine occurs in practically all healthy cells of plants, animals and microorganisms. Even though Morinda citrifolia has a negligible amount of free Xeronine, it contains appreciable amounts of the precursor of Xeronine, called Proxeronine. Further, Morinda citrifolia contains the inactive form of the enzyme Proxeronase which releases Xeronine from Proxeronine. A paper entitled, “The Pharmacologically Active Ingredient of Noni” by R. M. Heinicke of the University of Hawaii, indicates that Morinda citrifolia is “the best raw material to use for the isolation of xeronine,” because of the building blocks of Proxeronine and Proxeronase. These building blocks aid in the isolation and production of Xeronine within the body. The function of the essential nutrient Xeronine is fourfold.

First, Xeronine serves to activate dormant enzymes found in the small intestines. These enzymes are critical to efficient digestion, calm nerves, and overall physical and emotional energy.

Second, Xeronine protects and keeps the shape and suppleness of protein molecules so that they may be able to pass through the cell walls and be used to form healthy tissue. Without these nutrients going into the cell, the cell cannot perform its job efficiently. Without Proxeronine to produce Xeronine our cells, and subsequently the body, suffer.

Third, Xeronine assists in enlarging the membrane pores of the cells. This enlargement allows for larger chains of peptides (amino acids or proteins) to be admitted into the cell. If these chains are not used they become waste.

Fourth, Xeronine, which is made from Proxeronine, assists in enlarging the pores to allow better absorption of nutrients.

Each tissue has cells which contain proteins which have receptor sites for the absorption of Xeronine. Certain of these proteins are the inert forms of enzymes which require absorbed Xeronine to become active. Thus Xeronine, by converting the body's procollagenase system into a specific protease, quickly and safely removes the dead tissue from skin. Other proteins become potential receptor sites for hormones after they react with Xeronine. Thus the action of Morinda citrifolia in making a person feel well is probably caused by Xeronine converting certain brain receptor proteins into active sites for the absorption of the endorphin, the well being hormones. Other proteins form pores through membranes in the intestines, the blood vessels and other body organs. Absorbing Xeronine on these proteins changes the shape of the pores and thus affects the passage of molecules through the membranes.

Because of its many benefits, Morinda citrifolia has been known to provide a number of anecdotal effects in individuals having cancer, arthritis, headaches, indigestion, malignancies, broken bones, high blood pressure, diabetes, pain, infection, asthma, toothaches, blemishes, immune system failure, and others.

The compositions containing Morinda citrifolia may be in a form suitable for oral use, for example, as tablets, or lozenges, aqueous or oily suspensions, dispersible powders or granules, emulsions, syrups or elixirs. Compositions intended for oral use may be prepared according to any method known in the art for the manufacture of Morinda citrifolia compositions and such compositions may contain one or more agents selected from the group consisting of sweetening agents, flavoring agents, coloring agents and preserving agents. Tablets contain Morinda citrifolia in admixture with non-toxic pharmaceutically acceptable excipients which are suitable for the manufacture of tablets. These excipients may be for example, inert diluents, granulating and disintegrating agents, binding agents, and lubricating agents. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Aqueous suspensions contain the Morinda citrifolia in admixture with excipients suitable for the manufacture of aqueous suspensions. Such excipients are suspending agents, for example, sodium carboxymethyl-cellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitor monooleate, or condensation products of ethylene oxide with partial esters derived from fatty acids and hexitol anhydrides, for example polyethylene sorbitan monooleate.

Favorably, this invention provides a method of treating migraine headaches with a Morinda citrifolia-based formulation without any significant tendency to cause gastric side effects.

### Morinda Citrifolia-Based Naturaceutical Formulations and Methods of Treating Visual or Ocular Impairments

In the disclosure the term “eye disorder” or “visual impairment” shall refer to any type of eye disease, condition, attribute or disorder that affects vision. Examples of such eye disorders include glaucoma, diabetic retinopathy, retinitis pigmentosa, cataracts, age-related macular degeneration, night blindness, color blindness, and other vision affecting conditions.

In accordance with the present invention, a treatment in the form of eye drops, a dietary supplement, or other forms is used to treat one or more eye disorders. The treatment is preferably a naturaceutical specifically formulated with one or more processed Morinda citrifolia products that may comprise 100 percent of the formulation, or that may be combined with other ingredients, such as water, fruit juices, and other dietary, nutritional, or food supplements. Several formulations of the present invention naturaceutical formulation are provided below, although these are only to be considered as illustrative of some embodiments and are not to be construed as limiting. Other factors may also play in the treatment methods, such as varying the amount or dose of the naturaceutical used per treatment due to one or more factors, such as or including the type of eye disorder, the physical characteristics of the patient, etc.

The use of processed Morinda citrifolia has proven to be advantageous to treating variety of eye disorders by providing improved results. The processed Morinda citrifolia products function as, or comprise, various active ingredients and antibacterial properties, particularly useful in fighting visual impairments. Furthermore, the use of a processed Morinda citrifolia product has proven to inhibit macular degeneration. Introducing and internalizing processed Morinda citrifolia into the body treats macular degeneration in that the eye disorder is reduced upon the inclusion of the processed Morinda citrifolia product.

In one exemplary embodiment, the naturaceutical formulation comprises a processed Morinda citrifolia product in the form of a processed Morinda citrifolia leaf extract, which procedure or method of obtaining from the Indian Mulberry plant is described above.

In another exemplary embodiment, the naturaceutical formulation comprises a processed Morinda citrifolia product in the form of processed Morinda citrifolia fruit or puree juice in dilute or in concentrate form. As used herein, the term “Tahitian Noni®” refers to a product that includes the fruit juice processed from the fruit of the Morinda citrifolia L. In one embodiment, Tahitian Noni® includes reconstituted Morinda citrifolia L. fruit juice from pure juice puree of French Polynesia. Tahitian Noni® may also include other natural juices, such as a natural grape juice concentrate, a natural blueberry juice concentrate, and/or another natural juice concentrate. In a further embodiment, Tahitian Noni® is processed from dried or powdered Morinda citrifolia L. Tahitian Noni® may be obtained from Morinda, Inc. of Orem, Utah.

As stated, the present invention features a unique formulation and method of administering the same to treat and prevent visual impairments, or rather advances treatment of visual impairments by providing a naturaceutical composition or treatment formulated with one or more processed Morinda citrifolia products derived from the Indian Mulberry plant. The Morinda citrifolia product may be incorporated into various carriers or naturaceutical compositions suitable for in vivo treatment of a patient. For instance, the naturaceutical formulation may be embodied in a formulation that may be ingested orally, or the naturaceutical formulation may be embodied in an eye drop solution for direct placement on the eye, or the naturaceutical formulation may be embodied for intravenous injection or feeding, or any other methods or means for introducing the naturaceutical into the body of the individual for internalization by the individual as is appropriate and directed.

As discussed above, various eye disorders exist that affect vision. One such disorder of the eye is dry macular degeneration, wherein the retinal and choroidal blood vessels slowly disappear, as does the central retina. Alternatively, wet macular degeneration is the situation wherein abnormal blood vessels grow under the macular retina, leak, and cause scarring in the central retina. As such, the present invention features an exemplary embodiment method for treating macular degeneration, and particularly for inhibiting and preventing macular degeneration, as well as reversing existing or already occurring macular degeneration. The naturaceutical of the present invention functions to increase and normalize blood flow within the blood vessels of the eye, as well as to provide cellular enhancement to allow for better nutrient absorption and molecular transfer in and out of the cells in the blood vessels and tissues in the eye.

The present invention functions similarly to treat and prevent other known eye disorders, namely, glaucoma, diabetic retinopathy, retinitis pigmentosa, cataracts, age-related macular degeneration, night blindness, color blindness, and others. According to the present invention, internalizing the naturaceutical formulation comprising the one or more processed Morinda citrifolia products, as well as other ingredients if desired, serves to treat these abnormal occurrences in the eye.

The naturaceutical composition of the present invention comprises one or more of a processed Morinda citrifolia product present in an amount by weight between about 0.01 and 100 percent by weight, and preferably between 0.01 and 95 percent by weight. As stated, several exemplary embodiments of formulations are provided below, but are only intended to be exemplary as one ordinarily skilled in the art will recognize other formulations or compositions that may be manufactured and utilized comprising the processed Morinda citrifolia product.

The processed Morinda citrifolia product is the active ingredient or contains one or more active ingredients, such as Quercetin and Rutin, and others, for treating and relieving existing visual impairments, as well as reducing the onset potential of visual impairments. Active ingredients may be extracted out using various alcohol or alcohol-based solutions, such as methanol, ethanol, and ethyl acetate, and other alcohol-based derivatives using any known process in the art. The active ingredients of Quercetin and Rutin are present in amounts by weight ranging from 0.01-10 percent of the total formulation or composition. These amounts may be concentrated as well into a more potent concentration in which they are present in amounts ranging from 10 to 100 percent.

The naturaceutical composition comprising Morinda citrifolia may be prepared using any known means in the art. In addition, since the naturaceutical composition will most likely be consumed orally, it may contain one or more agents selected from the group consisting of sweetening agents, flavoring agents, coloring agents, preserving agents, and other medicinal agents as directed.

The present invention further features several methods of administering the naturaceutical composition to a mammal for the treatment and relief of visual impairments and to help prevent or reduce the likelihood or onset potential of visual impairments. Inn one exemplary embodiment, the method for administering a naturaceutical, or the method for treating and preventing visual impairments comprises the steps of (a) formulating a naturaceutical composition comprising in part a processed Morinda citrifolia product present in an amount between about 0.01 and 95 percent by weight, wherein the composition may also comprise a carrier, such as water or purified water, and other natural or artificial ingredients; (b) introducing the naturaceutical composition into the body, such that the Morinda citrifolia is sufficiently internalized; (c) repeating the above steps as often as necessary to provide an effective amount of the processed Morinda citrifolia to the tissues of the body of the patient to reduce vasoconstriction.

In an exemplary embodiment, the step of introducing the naturaceutical composition into the body comprises ingesting the composition orally. Ingesting the naturaceutical orally means the naturaceutical composition may be formulated as a liquid, gel, solid, or some other type that would allow the composition to be quickly digested and concentrated within the body of the user. It is important to note that the step of administering the naturaceutical composition should be carried out in an effective manner so that the greatest concentration of naturaceutical composition, and particularly the processed Morinda citrifolia product, is absorbed into the tissues of the patient's body. For the naturaceutical composition to take effect, it must be sufficiently internalized. Once sufficiently internalized, it may then begin to function to treat or relieve existing visual impairments and their associated conditions or symptoms, as well as to reduce the onset potential visual impairments as described above.

In one exemplary embodiment, the naturaceutical composition is administered by taking between 1 teaspoon and 2 oz., and preferably 2 oz., of the naturaceutical composition every two hours each day, or at least twice a day. Also, the naturaceutical composition is to be taken on an empty stomach, meaning at a period of time at least two hours prior to consumption of any food or drink. Following this, the naturaceutical composition is sufficiently allowed to absorb into the tissues of the body, and particularly within the region of the eye, to actively impact the tissue and blood vessels within the eye to assist proteins in forming pores through membranes in the blood vessels in the eye. Of course, one ordinarily skilled in the art will recognize that the amount of composition and frequency of use may vary from individual to individual.

In another embodiment, the method of introducing the naturaceutical may be done via an eye drop solution. Eye drop solutions are common in the art and the present invention contemplates formulation of an eye drop solution using any one of the salient ingredients known, wherein these ingredients are combined with one or more processed Morinda citrifolia products as described and taught herein.

In addition, the step of administering the naturaceutical composition may include injecting the composition into the body using an intravenous pump. This technique is advantageous as it would allow the composition to be localized in the area where it would have the most effect, or the area that would provide for the greatest concentration of the naturaceutical composition.

The treatment of visual impairments results from the affect that the processed Morinda citrifolia products, and/or the active ingredients found therein, namely Quercetin, Rutin, Xeronine, and the building blocks to Xeronine-Proxeronase and Proxeronine, described herein have on the blood vessels and tissues in the eye and region of the eye. Specifically, the processed Morinda citrifolia products, whether they be in the form of fruit juice, puree juice, leaf extracts, dietary fiber, oil, etc., function to convert certain receptor proteins and other enzymes into active sites for better and more efficient absorption of various nutrients. The processed Morinda citrifolia products also assist other proteins in forming pores through membranes in the blood vessels and tissues in the eye. As the Morinda citrifolia products are internalized into the body through the introduction of the naturaceutical formulation in which they are contained, they are absorbed by the various proteins in the body and go to work at facilitating the change in shape of the pores, thus positively or beneficially affecting the passage of molecules through the membranes. These unique affects specifically function to reduce and inhibit the overall effects that the above-mentioned factors have on the body in contributing to or directly causing visual impairments. Specifically, these affects, or rather the processed Morinda citrifolia products, function to increase or normalize blood flow and facilitate proper blood vessel functions, thus providing for a more natural and even flow of blood through the blood vessels existing within the eye.

The naturaceutical composition described above, and particularly the processed Morinda citrifolia products, also function to reduce and/or relieve many or all of the conditions and symptoms commonly associated with visual impairments, such as clouded vision, blurred vision, muscle strain, etc. As such, the present invention naturaceutical, and the methods of administering, further comprises treating such conditions or symptoms.

The following tables illustrate or represent some of the preferred formulations or compositions of the naturaceutical as contemplated by the present invention. It should be noted that these formulations are only intended as exemplary embodiments and are not to be construed as limiting in any way.

Formulation One

| Ingredients | Percent by Weight |
|---|---|
| Morinda citrifolia leaf hot water extract | 0.1-80% |
| water | 20-99.9% |

Formulation Two

| Ingredients | Percent by Weight |
|---|---|
| Morinda citrifolia leaf ethanol extract | 0.1-100% |

Formulation Three

| Ingredients | Percent by Weight |
|---|---|
| Morinda citrifolia leaf steam distillation extract | 0.1-100% |

Formulation Four

| Ingredients | Percent by Weight |
|---|---|
| Morinda citrifolia fruit juice or puree juice | 90-99.9% |
| Morinda citrifolia leaf water extract | 0.1-10% |

Formulation Five

| Ingredients | Percent by Weight |
|---|---|
| Morinda citrifolia fruit juice or puree juice | 90-99.9% |
| Morinda citrifolia leaf ethanol extract | 0.1-10% |

Formulation Six

| Ingredients | Percent by Weight |
|---|---|
| Morinda citrifolia fruit juice or puree juice | 90-99.9% |
| Morinda citrifolia leaf steam distillation extract | 0.1-10% |

Formulation Seven

| Ingredients | Percent by Weight |
|---|---|
| Morinda citrifolia leaf extract (water, ethanol, or | 0.1-10% |
| steam distillation) | |
| Morinda citrifolia fruit or puree juice or fruit/puree | 90-99.9% |
| juice concentrate | |
| Dietary supplement or food product | 90-99.9% |

Formulation Eight

| Ingredients | Percent by Weight |
|---|---|
| Morinda citrifolia puree juice or fruit juice | 100% |

Formulation Nine

| Ingredients | Percent by Weight |
|---|---|
| Morinda citrifolia fruit juice | 85-99.99% |
| water | 0.1-15% |

Formulation Ten

| Ingredients | Percent by Weight |
|---|---|
| Morinda citrifolia fruit juice | 85-99.99% |
| non-Morinda citrifolia-based fruit juices | 0.1-15% |

Formulation Eleven

| Ingredients | Percent by Weight |
|---|---|
| Morinda citrifolia fruit juice | 50-90% |
| water | 0.1-50% |
| non-Morinda citrifolia-based fruit juices | 0.1-30% |

Formulation Twelve

| Ingredients | Percent by Weight |
|---|---|
| Morinda citrifolia puree juice | 85-99.9% |
| water | 0.1-15% |

Formulation Thirteen

| Ingredients | Percent by Weight |
|---|---|
| Morinda citrifolia puree juice | 85-99.9% |
| non-Morinda citrifolia-based fruit juices | 0.1-15% |

Formulation Fourteen

| Ingredients | Percent by Weight |
|---|---|
| Morinda citrifolia puree juice | 50-90% |
| water | 0.1-50% |
| non-Morinda citrifolia-based fruit juices | 0.1-30% |

Formulation Fifteen

| Ingredients | Percent by Weight |
|---|---|
| Morinda citrifolia dietary fiber | 0.1-30% |
| water | 1-99.9% |
| non-Morinda citrifolia-based fruit juices | 1-99.9% |

Formulation Sixteen

| Ingredients | Percent by Weight |
|---|---|
| Morinda citrifolia dietary fiber | 0.1-30% |
| water | 1-99.9% |
| Morinda citrifolia fruit juice or puree juice | 1-99.9% |

Formulation Seventeen

| Ingredients | Percent by Weight |
|---|---|
| Morinda citrifolia puree juice concentrate or fruit | 100% |
| juice concentrate | |

Formulation Eighteen

| Ingredients | Percent by Weight |
|---|---|
| Morinda citrifolia fruit juice concentrate or puree | 85-99.99% |
| juice concentrate | |
| water | 0.1-15% |

Formulation Nineteen

| Ingredients | Percent by Weight |
|---|---|
| Morinda citrifolia fruit juice concentrate or puree | 1-10% |
| juice concentrate | |
| Eye drop solution | 90-99% |

In an exemplary embodiment or method, a person suffering from a visual impairment, and particularly diabetic retinopathy, or a person desiring to prevent the onset of visual impairments takes at least one ounce of a naturaceutical formulation comprising the ingredients in one of Formulations 1-7 in the morning on an empty stomach, and at least one ounce at night on an empty stomach, just prior to retiring to bed.

In another exemplary method, a person suffering from a visual impairment, or a person desiring to prevent the onset of visual impairments takes at least one ounce of a naturaceutical formulation as identified in Formulation Seven in the morning on an empty stomach, and at least one ounce at night on an empty stomach, just prior to retiring to bed.

The present invention further features taking a visual impairment medication concurrently with the naturaceutical formulation. Visual impairment medications used to treat existing eye disorders are well known in the art. Taking or administering the naturaceutical formulation, comprising one form or another of a processed Morinda citrifolia product as taught and described herein, functions to enhance the relief potential for the patient by increasing or enhancing the efficacy of the visual impairment medication, as well as providing the same benefits and advantages to the patient that are obtained directly from the naturaceutical formulation.

The following examples set forth and present the effects of Morinda citrifolia on both pre-existing visual impairments, on the symptoms and conditions associated with visual impairments, as well as the preventative effects of Morinda citrifolia against the onset of visual impairments. These examples are not intended to be limiting in any way, but are merely illustrative of the beneficial, advantageous, and remedial effects of the processed Morinda citrifolia products.

### EXAMPLE ONE

In the present example, a patient experiencing and diagnosed with an established visual impairment desires to treat the disorder with a non-prescription, over-the-counter remedy or preparation. Thus, to treat the disorder, the individual is given an identified, prescribed amount of a naturaceutical composition to consume orally, wherein the naturaceutical comprises 100% processed Morinda citrifolia fruit juice. The naturaceutical is administered in a safe, pre-determined amount and is administered intermittently a safe, pre-determined number of times, which, over time, alleviates the disorder and the symptoms associated therewith. In addition, the Morinda citrifolia-based naturaceutical is consumed by the patient on an empty stomach.

### EXAMPLE TWO

In the present example, a patient experiencing and diagnosed with an established visual impairment desires to treat the disorder with a non-prescription, over-the-counter remedy or preparation. Thus, to treat the disorder, the individual introduces a naturaceutical formulation in the form of an eye drop solution comprising a safe, pre-determined amount of a processed Morinda citrifolia product combined with various other saline ingredients adapted for introduction directly into the eye. The naturaceutical is administered in a safe, pre-determined amount and is administered intermittently a safe, pre-determined number of times, which, over time, alleviates the disorder and the symptoms associated therewith.

The present invention may be embodied in other specific forms without departing from its spirit of essential characteristics. The described embodiments are to be considered in all respects only al illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. A formulation adapted for treating and preventing visual impairments and for enhancing visual capacity, said formulation comprising:
a naturaceutical formulation comprising at least one processed Morinda citrifolia products.

2. The formulation of claim 1, wherein said processed Morinda citrifolia product comprises a processed Morinda citrifolia leaf hot water extract present in an amount by weight between about 0.1 and 50 percent.

3. The formulation of claim 1, wherein said processed Morinda citrifolia product comprises a processed Morinda citrifolia leaf ethanol extract present in an amount by weight between about 0.1 and 50 percent.

4. The formulation of claim 1, wherein said processed Morinda citrifolia product comprises a processed Morinda citrifolia leaf steam distillation extract present in an amount by weight between about 0.1 and 50 percent.

5. The formulation of claim 1, wherein said processed Morinda citrifolia product comprises Morinda citrifolia fruit juice.

6. The formulation of claim 1, wherein said processed Morinda citrifolia product comprises Morinda citrifolia puree juice.

7. The formulation of claim 1, wherein said processed Morinda citrifolia product comprises Morinda citrifolia puree juice concentrate.

8. The formulation of claim 1, wherein said processed Morinda citrifolia product comprises Morinda citrifolia fruit juice concentrate.

9. The formulation of claim 1, wherein said processed Morinda citrifolia product comprises Morinda citrifolia dietary fiber.

10. The formulation of claim 1, wherein said processed Morinda citrifolia product of said naturaceutical formulation further comprises an active ingredient Quercetin present in an amount between about 0.1 and 10 percent by weight.

11. The formulation of claim 1, wherein said processed Morinda citrifolia product is present in an amount between about 0.01 and 100 percent by weight.

12. The formulation of claim 11, wherein said processed Morinda citrifolia product further comprises Rutin as an additional active ingredient that synergistically works with said Quercetin to treat and prevent visual impairments and to enhance visual capacity.

13. The formulation of claim 12, wherein said Rutin is present in an amount between about 0.1 and 10 percent by weight.

14. The formulation of claim 1, wherein said naturaceutical is administered orally.

15. The formulation of claim 1, wherein said naturaceutical is administered intravenously.

16. The formulation of claim 1, wherein said naturaceutical is administered systemically.

17. The formulation of claim 1, wherein said naturaceutical further comprises other ingredients selected from the group consisting of a combination of processed Morinda citrifolia products, food supplements, dietary supplements, other fruit juices, other natural ingredients, flavorings, and any effective combination and effective concentration of these.

18. The formulation of claim 1, wherein said naturaceutical is formulated into an eye drop.

19. An eye drop for treating visual impairments and enhancing visual capacity, said eye drop comprising:
at least one processed Morinda citrifolia product present in an amount by weight between 0.1 and 50 percent;
a suitable carrier solution present in an amount by weight between 50 and 99.9 percent by weight.

20. The eye droop of claim 19, wherein said processed Morinda citrifolia product is selected from the group consisting of a processed Morinda citrifolia leaf hot water extract, a processed Morinda citrifolia leaf ethanol extract a processed Morinda citrifolia leaf steam distillation extract, processed Morinda citrifolia fruit juice, processed Morinda citrifolia puree juice, processed Morinda citrifolia fruit juice concentrate, processed Morinda citrifolia puree juice concentrate, and a processed Morinda citrifolia dietary fiber.

21. A method for treating and preventing visual impairments, said method comprising the steps of:
introducing a naturaceutical formulation into the body of an individual for internalization, said naturaceutical formulation comprising at least one processed Morinda citrifolia products; and
administering safe, pre-determined doses of said naturaceutical formulation at safe, pre-determined frequencies and on a continual basis for a safe, pre-determined amount of time.

22. The method of claim 21, wherein said step of introducing said naturaceutical formulation comprising introducing said naturaceutical directly into the eye of said individual, wherein said naturaceutical formulation is in the form of an eye drop.

23. The method of claim 21, wherein said step of introducing said naturaceutical formulation comprises orally administering a safe, pre-determined amount of said naturaceutical formulation for safe, pre-determined frequency for a safe, pre-determined duration.

24. The method of claim 21, wherein said at least one processed Morinda citrifolia product is selected from the group consisting of a processed Morinda citrifolia leaf hot water extract, a processed Morinda citrifolia leaf ethanol extract a processed Morinda citrifolia leaf steam distillation extract, processed Morinda citrifolia fruit juice, processed Morinda citrifolia puree juice, processed Morinda citrifolia fruit juice concentrate, processed Morinda citrifolia puree juice concentrate, and a processed Morinda citrifolia dietary fiber.

25. The method of claim 21, further comprising the step of supplementing said step of introducing a naturaceutical formulation with the administration of a visual impairment medication designed to treat one or more symptoms associated with a visual impairment, said visual impairment medication administered concurrently with said naturaceutical, wherein said naturaceutical increases the efficacy of said visual impairment medication.

26. The method of claim 23, wherein said safe, pre-determined amount is between about one teaspoon and about two ounces.

27. The method of claim 23, wherein said safe, pre-determined frequency comprises at least two times per day on an empty stomach.

28. The method of claim 23, wherein said safe, pre-determined duration of time comprises at least three consecutive days.

29. The method of claim 23, wherein said naturaceutical is orally administered in an amount between about 1 teaspoon and 2 ounces at least twice daily on an empty stomach each day.

30. A method for treating and preventing glaucoma visual impairments, said method comprising the steps of:
normalizing blood flow and enhancing cellular vitality in choroidal blood vessels by introducing a naturaceutical formulation into the body of a patient for internalization, said naturaceutical formulation comprising at least one processed Morinda citrifolia products adapted to treat one or more visual impairments; and
administering safe, pre-determined doses of said naturaceutical formulation at safe, pre-determined frequencies and on a continual basis for a safe, pre-determined amount of time.

31. The method of claim 30, wherein said step of introducing said naturaceutical formulation comprising introducing said naturaceutical directly into the eye of said individual, wherein said naturaceutical formulation is in the form of an eye drop.

32. The method of claim 30, wherein said step of introducing said naturaceutical formulation comprises orally administering a safe, pre-determined amount of said naturaceutical formulation for safe, pre-determined frequency for a safe, pre-determined duration.

33. The method of claim 30, wherein said at least one processed Morinda citrifolia product is selected from the group consisting of a processed Morinda citrifolia leaf hot water extract, a processed Morinda citrifolia leaf ethanol extract a processed Morinda citrifolia leaf steam distillation extract, processed Morinda citrifolia fruit juice, processed Morinda citrifolia puree juice, processed Morinda citrifolia fruit juice concentrate, processed Morinda citrifolia puree juice concentrate, and a processed Morinda citrifolia dietary fiber.

34. The method of claim 30, further comprising the step of supplementing said step of introducing a naturaceutical formulation with the administration of a visual impairment medication designed to treat one or more symptoms associated with a visual impairment, said visual impairment medication administered concurrently with said naturaceutical, wherein said naturaceutical increases the efficacy of said visual impairment medication.

35. A method for improving blood flow and enhancing cellular vitality in choroidal blood vessels, said method comprising the steps of:
orally administering at least one ounce of a naturaceutical formulation comprising processed Morinda citrifolia fruit juice on an empty stomach in the morning;
orally administering at least one ounce of said naturaceutical formulation prior to sleeping at night; and
repeating said steps of orally administering for continued consecutive days.

36. The formulation of claim 35, wherein said processed Morinda citrifolia product comprises a processed Morinda citrifolia leaf hot water extract present in an amount by weight between about 0.1 and 50 percent.

37. The formulation of claim 35, wherein said processed Morinda citrifolia product comprises a processed Morinda citrifolia leaf ethanol extract present in an amount by weight between about 0.1 and 50 percent.

38. The formulation of claim 35, wherein said processed Morinda citrifolia product comprises a processed Morinda citrifolia leaf steam distillation extract present in an amount by weight between about 0.1 and 50 percent.

39. The method of claim 35, wherein said naturaceutical formulation comprises:
processed Morinda citrifolia fruit juice present in an amount by weight of about 100 percent.

40. The method of claim 35, wherein said naturaceutical formulation comprises:
processed Morinda citrifolia fruit juice present in an amount by weight between about 85-99.99 percent; and
water present in an amount by weight between about 0.1-15 percent.

41. The method of claim 35, wherein said naturaceutical formulation comprises:
processed Morinda citrifolia fruit juice present in an amount by weight between about 85-99.99 percent; and
non-Morinda citrifolia-based fruit juices present in an amount by weight between about 0.1-15 percent.

42. The method of claim 35, wherein said naturaceutical formulation comprises:
processed Morinda citrifolia fruit juice present in an amount by weight between about 50-90 percent;
water present in an amount by weight between about 0.1-50 percent; and
non-Morinda citrifolia-based fruit juices present in an amount between about 0.1-30 percent.

43. A method for treating and preventing diabetic retinopathy, said method comprising the steps of:
introducing a naturaceutical formulation into the body of a patient for internalization, said naturaceutical formulation comprising at least one processed Morinda citrifolia leaf extract; and
administering safe, pre-determined doses of said naturaceutical formulation at safe, pre-determined frequencies and on a continual basis for a safe, pre-determined amount of time.

44. The method of claim 43, wherein said processed Morinda citrifolia leaf extract comprises a processed Morinda citrifolia leaf hot water extract.

45. The method of claim 43, wherein said processed Morinda citrifolia leaf extract comprises a processed Morinda citrifolia leaf ethanol extract.

46. The method of claim 43, wherein said processed Morinda citrifolia leaf extract comprises a processed Morinda citrifolia leaf steam distillation extract.

47. The method of claim 43, wherein said naturaceutical formulation further comprises a processed Morinda citrifolia product selected from the group consisting of processed Morinda citrifolia fruit juice selected from dilute and concentrate form, processed Morinda citrifolia puree juice selected from dilute and concentrate form, and processed Morinda citrifolia dietary fiber.

48. A method for treating and preventing retinitis pigmentosa, said method comprising the steps of:
introducing a naturaceutical formulation into the body of a patient for internalization, said naturaceutical formulation comprising at least one processed Morinda citrifolia product; and
administering safe, pre-determined doses of said naturaceutical formulation at safe, pre-determined frequencies and on a continual basis for a safe, pre-determined amount of time.

49. A method for treating and preventing cataracts, said method comprising the steps of:
introducing a naturaceutical formulation into the body of a patient for internalization, said naturaceutical formulation comprising at least one processed Morinda citrifolia product; and
administering safe, pre-determined doses of said naturaceutical formulation at safe, pre-determined frequencies and on a continual basis for a safe, pre-determined amount of time.

50. A method for inhibiting, preventing, and reversing macular degeneration, said method comprising the steps of:
introducing a naturaceutical formulation into the body of a patient for internalization, said naturaceutical formulation comprising at least one processed Morinda citrifolia product; and
administering safe, pre-determined doses of said naturaceutical formulation at safe, pre-determined frequencies and on a continual basis for a safe, pre-determined amount of time.

51. A method for improving visual capacity, said method comprising the steps of:
introducing a naturaceutical formulation into the body of a patient for internalization, said naturaceutical formulation comprising at least one processed Morinda citrifolia product; and
administering safe, pre-determined doses of said naturaceutical formulation at safe, pre-determined frequencies and on a continual basis for a safe, pre-determined amount of time.

52. A method for treating an existing visual impairment comprising the steps of:
administering a visual impairment medication to a patient;
administering a naturaceutical to said patient concurrently with said visual impairment medication, said naturaceutical comprising a processed Morinda citrifolia product that reduces and inhibits the breaking down or metabolizing of said visual impairment medication by inhibiting the medication breakdown functions in the body of said patient, thus increasing the efficacy of said visual impairment medication, said processed Morinda citrifolia product present in an amount by weight between 0.1 and 100 percent.
